(19) United States
(12) Patent Application Publication
(10) Pub. No.: US 2025/0353003 A1
(43) Pub. Date: Nov. 20, 2025

(54) HYDROPHOBIC CARTRIDGE FOR DIGITAL MICROFLUIDICS

(71) Applicant: INTEGRA MICROFLUIDICS CORPORATION, San Francisco, CA (US)

(72) Inventors: Mais J. JEBRAIL, Toronto (CA); Rohit LAL, San Francisco, CA (US)

(21) Appl. No.: 18/873,323

(22) PCT Filed: Jun. 9, 2023
(86) PCT No.: PCT/US2023/068210

§ 371 Date: Dec. 9, 2024

Related U.S. Application Data
(60) Provisional application No. 63/350,618, filed on Jun. 9, 2022.

(51) Int. Cl. B01L 3/00

(52) U.S. Cl. CPC B01L 3/502707; B01L 2200/12; B01L 2300/0645; B01L 2300/0887; B01L 2300/12; B01L 2300/165

(57) ABSTRACT

Methods and compounds are disclosed for making and/or forming hydrophobic cartridges for use with digital microfluidic (DMF) apparatuses. The hydrophobicity of DMF cartridges may be improved by mixing an effective amount of one or more fluorinated surfactants with the polymer or polycarbonate resins used to form the cartridge. Methods for preparing a molding compound for use in a DMF cartridge may include heating a compound of polycarbonate and a fluorinated surfactant for a predetermined time period.

### CLAIM OF PRIORITY

This patent application claims priority to U.S. provisional patent application No. 63/350,618, titled “HYDROPHOBIC CARTRIDGE FOR DIGITAL MICROFLUIDICS” and filed on Jun. 9, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The disclosure relates to digital microfluidic devices and associated fluid manipulation and extraction devices, and methods of manufacturing them.

### BACKGROUND

Digital microfluidics (DMF) is a powerful technique for simple and precise manipulation of microscale droplets of fluid. DMF has rapidly become popular for chemical, biological, and medical applications, as it allows straightforward control over multiple reagents (no pumps, valves, or tubing required), facile handling of both solids and liquids (no channels to clog), and compatibility with even troublesome reagents (e.g., organic solvents, corrosive chemicals) because hydrophobic surfaces (typically treated with one or more hydrophobic coatings) in contact with the droplets of fluid are chemically inert. Conventional DMF devices use relatively large electric fields selectively applied to an array of electrodes to manipulate the droplets. The generation and control of these electric fields requires specialized and complex circuitry capable of withstanding the relative high voltages.

However, the hydrophobic coatings require application through additional processing steps. Thus, there is a need for hydrophobic surfaces attained with few processing steps.

### SUMMARY OF THE DISCLOSURE

Described herein are methods and components are disclosed for making and/or forming hydrophobic cartridges, such as (but not limited to) those for use with any digital microfluidic (DMF) apparatus. The hydrophobicity of DMF cartridges may be improved by mixing a fluorinated surfactants with polymer or polycarbonate resins used to form DMF cartridges.

For example, described herein are microfluidics cartridges comprising: a first plate having a first side and a second side; and a second plate; wherein the first plate and the second plate are secured opposite and parallel to each other with an air gap therebetween, further wherein at least the first plate comprises an injection molding compound including: a polycarbonate, and an effective amount of a fluorinated surfactant to increase hydrophobicity of the first plate.

In general, any of these cartridges may be digital microfluidics (DMF) cartridges. For example, the cartridge may be for use with a DMF apparatus and may include a first (e.g., top) plate having a first side and a second side, a ground electrode disposed on the first side of the top plate, a second (e.g., bottom) plate, wherein at least the top plate and the bottom plate comprise an injection molding compound that includes a polycarbonate, and an effective amount of a fluorinated surfactant to increase hydrophobicity of the top plate and the bottom plate, and a frame, configured to separate the top plate from the bottom plate and form an air gap therebetween, wherein the first side of the top plate is disposed toward the frame.

In any of the cartridges disclosed herein, an effective amount of the fluorinated surfactant may be about 0.4% by weight of the polycarbonate. In any of the cartridges, the fluorinated surfactant may be configured to bloom on surfaces of the top plate and the bottom plate. Furthermore, in any of the cartridges disclosed herein the fluorinated surfactant may be configured to increase a deionized water contact angle to greater than about 90 degrees with respect to the top plate and the bottom plate.

In any of the cartridges disclosed herein, the fluorinated surfactant is a trifluoroethyl methacrylate (TFMA). Furthermore, the injection molding compound further may include a colorant in an amount of about 4% by weight of the polycarbonate. The colorant may be e colorant is Clariant Mevopur NC7M820049.

In any of the cartridges disclosed herein, the ground electrode may be disposed on a surface of the top plate. Furthermore, in any of the cartridges the ground electrode may be formed from a non-transparent material, a conductive ink, silver nanoparticles, or a combination thereof.

In any of the cartridges disclosed herein, the polycarbonate may be a medical-grade polycarbonate resin. In any of the cartridges disclosed herein, the fluorinated surfactant is Cytonix FluoroPel TFMA-6.

Example methods for preparing a hydrophobic injection molding compound for use in a cartridge apparatus are disclosed. The example methods may include grinding a fluorinated surfactant into a powder, forming a compound by combining together the fluorinated surfactant and a plurality of polycarbonate pellets, actively mixing the compound for at least five minutes, and heating the compound to about 115 degrees Celsius for at least four hours after actively mixing.

In any of the methods described herein, the fluorinated surfactant may be in an amount of about 0.4% by weight of the plurality of polycarbonate pellets. Furthermore, any of the methods may include adding a colorant in an amount of about 4% by weight of the plurality of polycarbonate pellets to the compound, wherein actively mixing further comprises actively mixing the colorant with the plurality of polycarbonate pellets. The colorant may be Clariant Mevopur NC7M820049. In any of the methods, the colorant may be added prior to heating the compound.

In any of the methods described herein, the fluorinated surfactant may be a trifluoroethyl methacrylate (TFMA). In any of the methods described herein, the fluorinated surfactant may be Cytonix FluoroPel TFMA-6.

In any of the methods, the plurality of polycarbonate pellets may be medical-grade polycarbonate pellets. Furthermore, in any of the methods, the fluorinated surfactant may be a dry melt hydrophobic additive. In any of the methods described herein, actively mixing the compound may occur at an ambient temperature.

Other example methods may include receiving a compound of a fluorinated surfactant and polycarbonate pellets, heating and controlling a temperature of the compound to a temperature of about 250 degrees Celsius and injecting the compound into an injection mold. In any of the methods described herein, the fluorinated surfactant may be in an amount of about 0.4% by weight of the polycarbonate pellets. Furthermore, in any of the methods, the fluorinated surfactant maybe a trifluoroethyl methacrylate (TFMA).

In any of the methods described herein, the compound may include a colorant in an amount of about 4% by weight of the polycarbonate pellets. Furthermore, the colorant may be Clariant Mevopur NC7M820049.

Any of the methods described herein may further include aging the cartridge for a period of not less than two days after injection prior to using the cartridge. In any of the methods, the polycarbonate pellets are medical-grade polycarbonate pellets. Furthermore, the fluorinated surfactant may be a dry melt hydrophobic additive. In any of the methods described herein, the compound may be heated to a temperature of about 115 degrees Celsius for a period of about four hours prior to being received.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a flowchart depicting an example method preparing a compound for use in manufacturing of a cartridge for use with any microfluidic apparatus.

FIG. 2 is a flowchart depicting an example method for injection molding an article for use with a microfluidics apparatus.

FIG. 3 shows an exploded view of a simplified representation of a microfluidics cartridge.

FIG. 4 shows an exploded view of another example microfluidics cartridge.

FIG. 5 shows three different example images showing contact angles for deionized water with a conventional polymer or polycarbonate resin.

FIG. 6 shows three different example images showing contact angles for deionized water with a polymer or polycarbonate resin similar to the formulation (e.g., that includes TFMA) described with respect to FIG. 1.

### DETAILED DESCRIPTION

Methods and compounds are disclosed for making and/or forming hydrophobic cartridges for use with any microfluidic apparatus. The hydrophobicity of a microfluidics cartridges may be improved by mixing one or more fluorinated surfactants with polymer or polycarbonate resins prior to heating the resulting compound for use in an injection molding process.

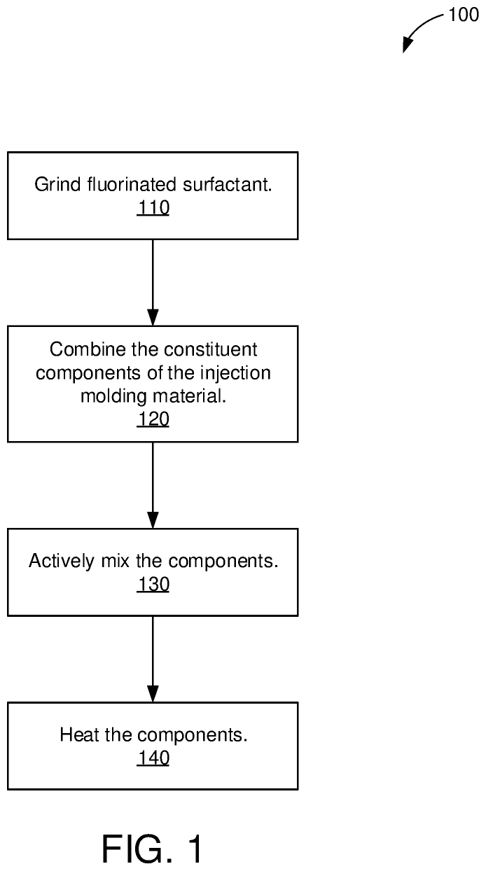

FIG. 1 is a flowchart depicting an example method 100 preparing a compound for use in manufacturing of a cartridge for use with any microfluidics apparatus, including but not limited to a digital microfluidics (DMF) apparatus. Injection molding is described herein, buy any other feasible method may be used to form a cartridge. Conventional injection molding techniques may use a polymer as a primary material. Hydrophobicity of the primary material may be increased by an addition of a fluorinated surfactant. Additionally, one or more colorants may also be added to the primary material. The colorant may not affect hydrophobicity but may allow a cosmetic tinting of the cartridge.

The method 100 may begin in block 110 as the fluorinated surfactant is ground. In some examples, the fluorinated surfactant may be in pellet form. In some other examples, the fluorinated surfactant may have an irregular (e.g., non-uniform) size and shape. Thus, the grinding may provide a more uniform size and shape of the fluorinated surfactant. This uniform size and shape may allow a more even distribution of the surfactant within the primary material. In some examples, the fluorinated surfactant may be ground into a fine powder.

In some variations, the fluorinated surfactant may be a trifluoroethyl methacrylate (TFMA). A non-limiting example of TFMA may be FluroPel TFMA-6 from Cytonix LLC. Other TFMA surfactants are possible. In some examples, the TFMA may be a dry melt hydrophobic additive.

Next, in block 120 constituent components of the injection molding material are combined. The constituent components of the injection molding material may include the primary material, the fluorinated surfactant, and (optionally) a colorant. To ensure a consistent product with uniform hydrophobic characteristics, amounts of each of the components of the injection molding material may be determined with respect to weight of the primary material.

The primary material may be any polymer or polymer-like material that is suitable for injection molding. In some examples, the primary material may be polycarbonate resin. A non-limiting example of a polycarbonate resin may be a Makrolon 2458 resin. In some variations, the polycarbonate resin may be any feasible medical-grade polycarbonate resin. The colorant may be any feasible colorant compatible with the primary material and the fluorinated surfactant. An example colorant may be Clariant Mevopur NC7M820049.

The amount of the fluorinated surfactant may be about 0.4% by weight with respect to the weight of the primary material. In some examples, about 0.4% by weight with respect to the weight of the primary material may be an effective amount of fluorinated surfactant to increase hydrophobicity of a cartridge formed from such a compound. However, in some variations, an effective amount of fluorinated surfactant used may be more or less than 0.4% by weight of the primary material.

The amount of the colorant may be about 4% by weight with respect to the weight of the primary material. In some examples, about 4% by weight with respect to the weight of the primary material may be an effective amount of colorant to tint a cartridge formed from such a compound. In some variations, an effective amount of the colorant used may be more or less than 4% by weight of the primary material.

Next, in block 130, the components are actively mixed. For example, the components noted in block 120 may be mixed for at least a minimum time period. An example minimum time period may be five minutes, however any other feasible time period that evenly distributes the components of the compound may be used. In some variations, the mixing may be at an ambient or room temperature. An example ambient or room temperature may be 10 degrees Celsius, however other ambient temperatures may be used.

Next in block 140, the components are heated. In some examples, the components may be heated for a minimum amount of time at a controlled temperature. For example, a minimum time period to heat the components may be about four hours, however other minimum time periods may be used. The controlled temperature may be about 115 degrees Celsius, however, in some other variations other controlled temperatures may be used.

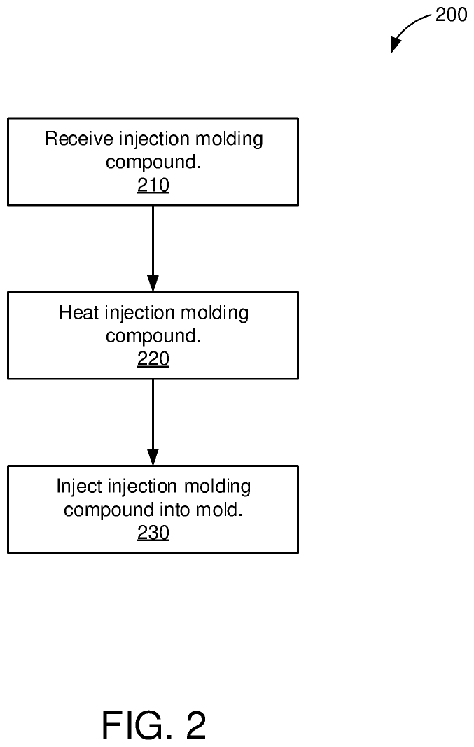

FIG. 2 is a flowchart depicting an example method 200 for injection molding an article for use with an apparatus. The method 200 may begin in block 210 as an injection molding compound (e.g., the mixed components of FIG. 1) is received. For example, the compound described with respect to FIG. 1 (e.g., a polymer, a fluorinated surfactant in an amount of about 0.4% by weight of the polymer, and a colorant in an amount of about 4% by weight of the polymer) may be received by a hopper of any suitable injection molding equipment.

Next, in block 220 the injection molding compound is heated. In some examples, the injection molding compound may be heated to about 250 degrees Celsius. In some variations the injection molding compound may be heated to a temperature greater than 250 degrees Celsius, but only for a limited time period. In some examples, heating the injection molding compound to a temperature of about 250 degrees Celsius may be sufficient to liquify the molding compound but not high enough to damage or affect the hydrophobic characteristics of the TFMA included in the compound.

Next, in block 230, the injection molding compound is injected into a mold. In some examples, the mold may be for all or part of a cartridge. By molding all or part of a DMF cartridge with a TFMA-bearing compound, the respective cartridge may have increased hydrophobicity compared to cartridges made without TFMA.

In some variations, the injection molded parts may be aged for a predetermined time period after injection into the mold. In some examples, the predetermined time period may be as little as two days and, in some cases, up to ten days after being injection molded. Waiting for the predetermined time period to pass may allow the TFMA to “bloom” on outer surfaces of the injection molded parts. The aging process may allow the surface of the injection molding parts to develop maximum hydrophobicity.

One or more components of a cartridge for use with any feasible microfluidics apparatus may be injection molded as described with respect to FIG. 2, with an injection molding compound as described with respect to FIG. 1. The resulting cartridge may have one or more hydrophobic surfaces. Hydrophobic surfaces may increase performance of the associated cartridge by reducing surface fouling, for example.

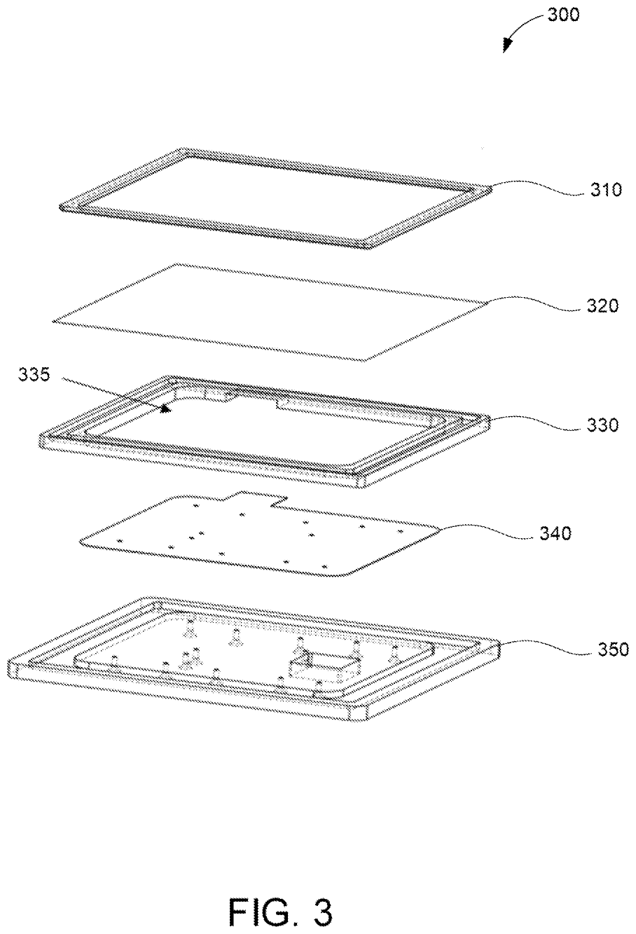

FIG. 3 shows an exploded view of a simplified representation of a cartridge 300, configured as a DMF cartridge. Exemplary DMF cartridges and apparatus are described in U.S. patent application Ser. No. 16/259,984, filed Jan. 28, 2019, now U.S. Pat. No. 11,311,882, which is commonly assigned, the disclosure of which is incorporated by reference herein in its entirety.

The DMF cartridge 300 may include an upper frame 310, a top plate 320, a tensioning frame 330, a bottom plate 340, and a base 350. Other cartridges 300 may include more or fewer components than as described in FIG. 3. In some variations, the components of the DMF cartridge 300 may be arranged differently than as shown and described in FIG. 3.

The top plate 320 may be coupled to the upper frame 310. In some variations, the top plate 320 may include a conductive material that may function as an electrode (for example, as a ground electrode). In some examples, the electrode may be formed from a non-transparent material, a conductive ink, and/or silver nanoparticles. The combination of the upper frame 310 and the top plate 320 may be coupled to the tensioning frame 330. The bottom plate 340 may also be coupled to the tensioning frame 330. In some examples, the bottom plate 340 may be thin and relatively flexible. In some variations, the tensioning frame 330 may hold the thin and flexible bottom plate 340 flat by providing a uniform outward (with reference to a center of the bottom plate 340) tension to the bottom plate 340.

The tensioning frame 330 may be mounted (coupled) to the base 350. The base 350 may facilitate temporarily mounting and/or affixing the DMF cartridge 300 to a DMF apparatus (not shown). The tensioning frame 330 may provide or form an air gap 335 between the top plate 320 and the bottom plate 340. In addition, one or more openings may be provided in the top plate 320 and/or the bottom plate 340 to allow a user to introduce specimens, reagents, or the like to the air gap 335. The specimens, reagents and other chemicals may be used to provide analysis or assay of any feasible specimen.

One or more components of the cartridge 300 may be formed from the compound material described with respect to FIG. 1 and injection molded as described with respect to FIG. 2. Thus, the surfaces of the components of the DMF cartridge 300 may be hydrophobic. In particular, the top plate 320 and the bottom plate 340 may be hydrophobic which may reduce any surface fouling associated with DMF activities within the air gap 335. In addition, the DMF cartridge 300 may include one or more openings to allow specimens, reagents, liquids, and the like to be introduced into the air gap 335. Any of these apparatuses may include an air gap between the first plate and the second plate. The air gap may be configured to hold a droplet between the plates, e.g., contacting both plates or contacting at least one (e.g., bottom) plate. The air gap may be between about 0.1 mm and about 7 mm (e.g., between about 0.2 mm and about 5 mm, between about 0.2 mm and about 4 mm, between about 0.3 mm and about 5 mm, between about 0.2 mm and about 3.5 mm, between about 0.2 mm and about 3 mm, etc.).

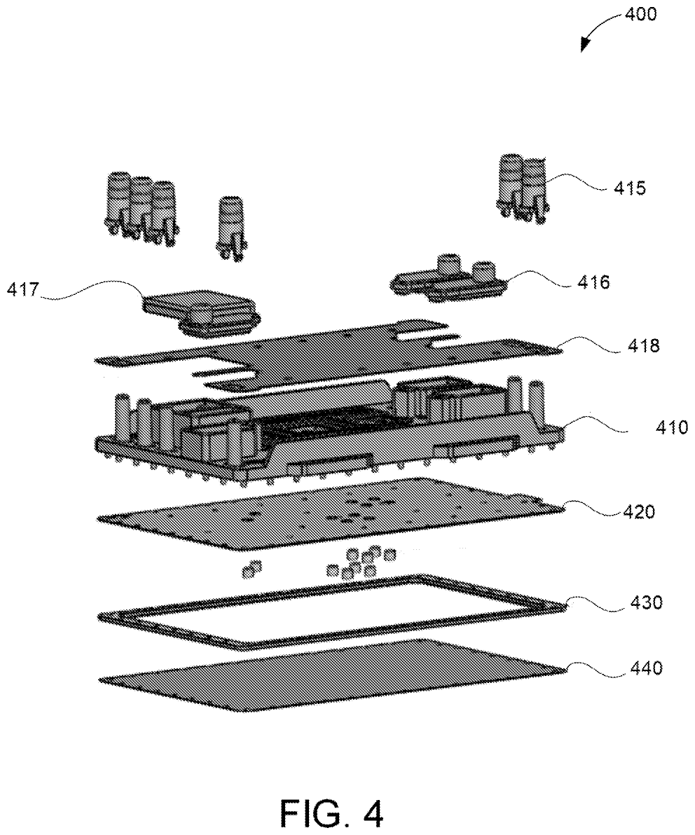

FIG. 4 shows an exploded view of another example DMF cartridge 400. The DMF cartridge 400 may include a body 410, a top plate 420, a frame 430 and a bottom plate 440. The body 410 may include one or more microfluidic channels and/or chambers (not shown) for dispensing or receiving fluid into/out of an air gap (not shown), which is bounded between the top plate 420 and the bottom plate 440. In some examples, a plurality of connectors 415 may allow solvents, reagents, specimens and the like to be introduced into the air gap of the DMF cartridge 400.

In some examples, one or more reservoirs 416 may be affixed to the body 410. The reservoirs 416 may be used to store reagents or other solutions that may be used during analysis and/or assay. Furthermore, one or more waste receptacles 417 may also be affixed to the body 410. The waste receptacles 417 may be used to receive and/or store waste liquids produced during analysis and/or assay. For example, spent reagents or wash byproducts may be stored in the waste receptacles 417. The body 410 may also include a protective film 418.

The top plate 420, the frame 430, and the bottom plate 440 may form the air gap of the DMF cartridge 400. In some examples, the top plate 420, the frame 430, and the bottom plate 440 may be formed from the compound material described with respect to FIG. 1 and injection molded as described with respect to FIG. 2. Thus, the top plate 420, the frame 430, and/or the bottom plate 440 may be formed from a hydrophobic material. Note that for convenience we refer to “top” and “bottom” plates herein, however these may more accurately be referred to herein as first and second plates, and may be arranged in any orientation (top/bottom), etc.

In some variations, the top plate 420 may include a conductive material that may function as an electrode (for example, as a ground electrode). In some variations, the bottom plate 440 may be thin and flexible and held in tension by at least the frame 430.

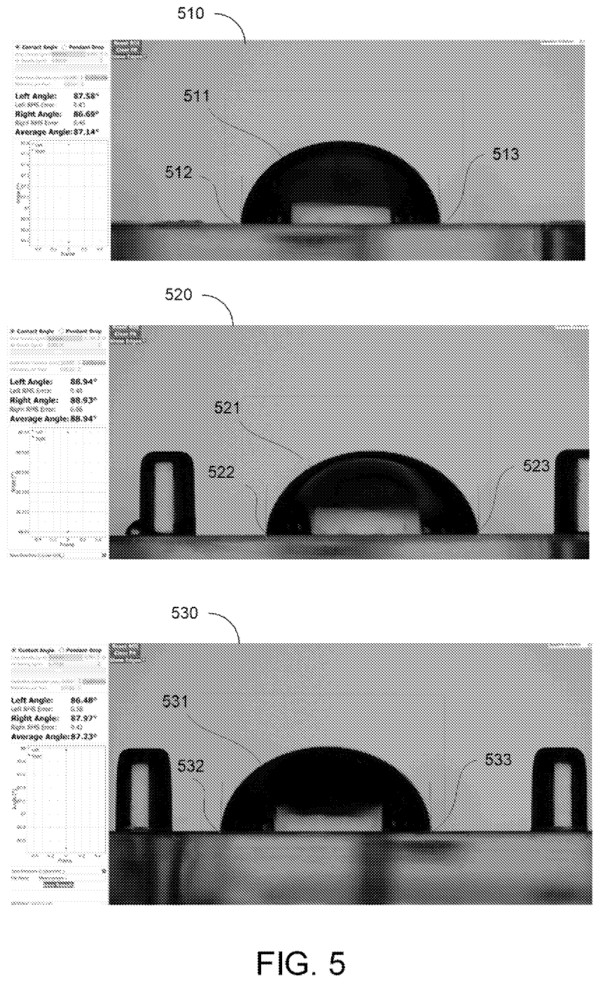

FIG. 5 shows three different example images showing contact angles for deionized water with a conventional polymer or polycarbonate resin. Deionized water contact angles can provide an indication of hydrophobicity. Generally, the greater the water contact angle, the greater the hydrophobicity of the polymer or polycarbonate resin.

In image 510, a first water droplet 511 has a left contact angle 512 of about 87.58 degrees, a right contact angle 513 of about 86.69 degrees, and an average contact angle of about 87.14 degrees. In image 520, a second water droplet 521 has a left contact angle 522 of about 88.94 degrees, a right contact angle 523 of about 88.93 degrees, and an average contact angle of about 88.94 degrees. In image 530, a third water droplet 531 has a left contact angle 532 of about 86.48 degrees, a right contact angle 533 of about 87.97 degrees, and an average contact angle of about 87.23 degrees. Thus, it can be seen that an average water contact angle for a conventional polymer or polycarbonate resin can be about 87.77 degrees.

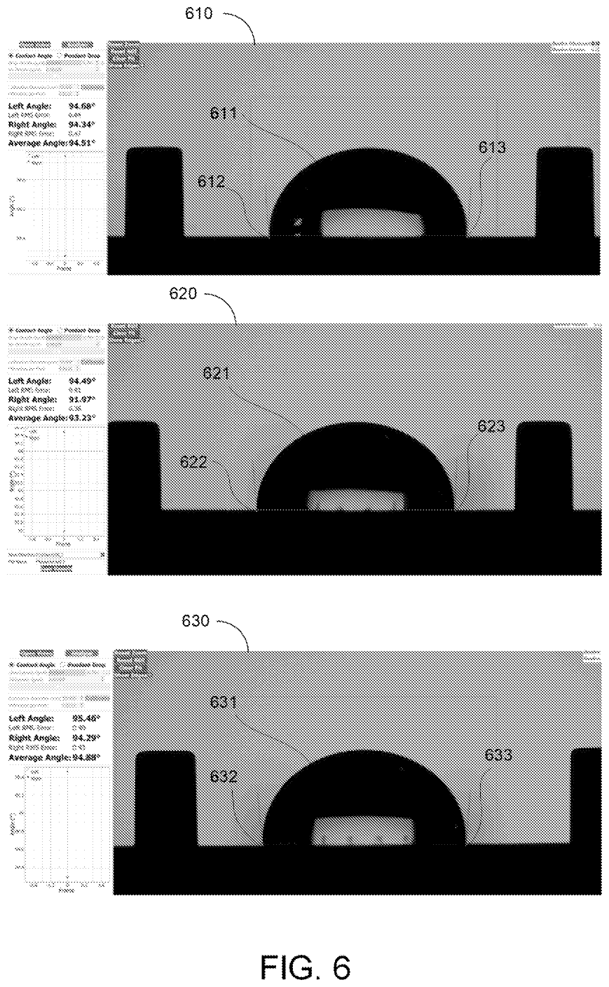

FIG. 6 shows three different example images showing contact angles for deionized water with a polymer or polycarbonate resin similar to the formulation (e.g., that includes TFMA) described with respect to FIG. 1.

In image 610, a first water droplet 611 has a left contact angle 612 of about 94.68 degrees, a right contact angle 613 of about 94.34 degrees, and an average contact angle of about 94.51 degrees. In image 620, a second water droplet 621 has a left contact angle 622 of about 94.49 degrees, a right contact angle 623 of about 91.97 degrees, and average contact angle of about 93.23 degrees. In image 630, a third water droplet 631 may have a left contact angle 632 of 95.46 degrees, a right contact angle 633 of 94.29 degrees, and an average contact angle of 94.88 degrees.

Thus, for deionized water in contact with a polymer or polycarbonate resin that includes TFMA, the overall average for a water contact angle can be about 94.20 degrees. This overall average water contact angle is greater than the overall average water contact angle for conventional polymers or polycarbonate resins. Therefore, polymers or polycarbonate resins with TFMA may have a relatively higher hydrophobicity.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A microfluidics cartridge, the cartridge comprising:
a first plate having a first side and a second side;
a second plate;
wherein the first plate and the second plate are secured opposite and parallel to each other with an air gap therebetween,
further wherein at least the first plate comprises an injection molding compound including: a polycarbonate, and an effective amount of a fluorinated surfactant to increase hydrophobicity of the first plate.

2. The cartridge of claim 1, wherein the effective amount of the fluorinated surfactant is about 0.4% by weight of the polycarbonate.

3. The cartridge of claim 1, wherein the fluorinated surfactant is configured to bloom on a surface of the first side of the plate within the air gap.

4. The cartridge of claim 1, wherein the fluorinated surfactant is configured to increase a deionized water contact angle to greater than about 90 degrees with respect to the top plate and the bottom plate.

5. The cartridge of claim 1, wherein the fluorinated surfactant is a trifluoroethyl methacrylate (TFMA).

6. The cartridge of claim 1, wherein the injection molding compound further includes a colorant in an amount of about 4% by weight of the polycarbonate.

7. The cartridge of claim 6, wherein the colorant is Clariant Mevopur NC7M820049.

8. The cartridge of claim 1, wherein the fluorinated surfactant is configured to increase a hydrophobicity of the first plate.

9. The cartridge of claim 1, wherein the polycarbonate is a medical-grade polycarbonate resin.

10. The cartridge of claim 1, wherein the fluorinated surfactant is Cytonix FluoroPel TFMA-6.

11. A cartridge for use with a digital microfluidics (DMF) apparatus, the cartridge comprising:
a top plate having a first side and a second side;
a ground electrode disposed on the first side of the top plate;
a bottom plate, wherein at least the top plate and the bottom plate comprise an injection molding compound including:
a polycarbonate; and
an effective amount of a fluorinated surfactant to increase hydrophobicity of the top plate and the bottom plate; and
a frame, configured to separate the top plate from the bottom plate and form an air gap therebetween, wherein the first side of the top plate is disposed toward the frame.

12. The cartridge of claim 11, wherein the effective amount of the fluorinated surfactant is about 0.4% by weight of the polycarbonate.

13. The cartridge of claim 11, wherein the fluorinated surfactant is configured to bloom on surfaces of the top plate and bottom plate.

14. The cartridge of claim 11, wherein the fluorinated surfactant is configured to increase a deionized water contact angle to greater than about 90 degrees with respect to the top plate and the bottom plate.

15. The cartridge of claim 11, wherein the fluorinated surfactant is a trifluoroethyl methacrylate (TFMA).

16. The cartridge of claim 11, wherein the injection molding compound further includes a colorant in an amount of about 4% by weight of the polycarbonate.

17. The cartridge of claim 16, wherein the colorant is Clariant Mevopur NC7M820049.

18. The cartridge of claim 11, wherein the fluorinated surfactant is configured to increase a hydrophobicity of the top plate and the bottom plate.

19. The cartridge of claim 11, wherein the ground electrode is disposed on a surface of the top plate.

22. The cartridge of claim 11, wherein the ground electrode is formed from a non-transparent material, a conductive ink, silver nanoparticles, or a combination thereof.

21. The cartridge of claim 11, wherein the polycarbonate is a medical-grade polycarbonate resin.

22. The cartridge of claim 11, wherein the fluorinated surfactant is Cytonix FluoroPel TFMA-6.

23.-42. (canceled)
